(19) United States
(12) Patent Application Publication
(10) Pub. No.: US 2003/0119177 A1
(43) Pub. Date: Jun. 26, 2003

(54) Sample chip

(76) Inventors: Lewis Gruber, Chicago, IL (US); Dan Mueth, Chicago, IL (US)

Correspondence Address: SONNENSCHEIN NATH & ROSENTHAL, P.O. BOX 061080, WACKER DRIVE STATION, CHICAGO, IL, 60606-1080, US

(21) Appl. No.: 10/294,599

(22) Filed: Nov. 15, 2002

Related U.S. Application Data
(60) Provisional application No. 60/332,363, filed on Nov. 15, 2001.

(51) Int. Cl. C12M 1/34

(52) U.S. Cl. 435/287.2

(57) ABSTRACT

A sample chip includes a body portion; and a cover portion disposed on the body portion; wherein an upper surface of the body portion includes a plurality of microchannels in which objects are introduced for examination and manipulation by optical traps. In one embodiment, at least one of the microchannels includes a barrier which holds the objects so that they can be held and manipulated by the optical traps. In another embodiment, at least one of the microchannels includes a sample chamber at which the barrier is disposed. The number of microchannels and their configuration can vary, and the microchannels may intersect, the sample chamber being disposed at the intersection. The barrier includes at least one of a plurality of barrier structures which are integrally formed or removably disposed in the sample chamber. The barrier structures can take different shapes and can be in any combination of shapes.

The present invention claims priority from U.S. provisional application No. 60/332,363, dated Nov. 15, 2001, which is herein incorporated by reference.

### BACKGROUND OF THE INVENTION

1. Field of the Invention

The present invention relates generally to a sample chip which is used as part of a system for controlling and manipulating small objects using laser-generated optical traps.

2. Discussion of the Related Art

Conventional sample chips are used to introduce and flow solutions containing samples or other materials used in an experiment or process. The sample chip includes a plurality of microchannels through which a solution is introduced via one or more inlet portions, and discharged via one or more outlet portions. The solution includes samples or other materials having a plurality of objects (i.e., cells, beads, workpiece, etc.) which may be examined or acted upon in the microchannels of the sample chip, by a variety of means. After examination, the objects flow with the solution to be discharged via the outlet portion of the microchannel.

It is also known in the art, that the examination and manipulation of an object can be performed by holding or moving the object or sample using an optical “trap”, also called an optical “tweezer” as taught by Ashkin in U.S. Pat. No. 4,893,886. Ashkin teaches the generation and use of optical traps to manipulate biological material.

It is also know in the art to optically trap multiple objects with multiple, simultaneously-generated and simultaneously-controlled, independently movable, optical traps. (See generally U.S. Pat. No. 6,055,106 issued to Grier & Dufresne. These patents are hereby incorporated by reference in this application in order to more fully describe the state of the art to which this invention pertains.) Sophisticated manipulations of objects by optical trapping with control of the traps in three dimensions may be performed, for example, by using the BioRyx™ 200 system (available from Arryx, Inc., Chicago, Ill.).

One explanation of the mode of operation of an optical trap is that the gradient forces of a focused beam of light illuminating a object, trap that object, based on the dielectric constant of the object. An object having a dielectric constant higher than that of the surrounding medium will experience a force in the direction of the region of an optical trap where the light intensity and electric field is the highest.

Other types of optical traps that may be used to optically manipulate objects include, but are not limited to, optical vortices, optical bottles, optical rotators and light cages. An optical vortex produces a gradient surrounding an area of zero electric field which is useful to manipulate objects with dielectric constants lower than the surrounding media, or which are reflective, or other types of objects which are repelled by an optical trap. To minimize its energy, such an object will move to the region where the electric field is the lowest, namely the zero electric field area at the focal point of an appropriately shaped laser beam. The optical vortex provides an area of zero electric field much like the hole in a doughnut (toroid). The optical gradient is radial with the highest electric field at the circumference of the doughnut. The optical vortex detains a small object within the hole of the doughnut. The detention is accomplished by slipping the vortex over the small object along the line of zero electric field.

In general, optical traps are used to either manipulate materials such as in the area of constructing arrays of dielectric objects, or manipulating and/or investigating biological or chemical materials, as taught in pending U.S. patent application Ser. No. 09/886,802, filed Jun. 20, 2001, entitled “Configurable Dynamic Three Dimensional Array”, which is herein incorporated by reference.

Thus, objects in a solution are introduced into a sample chip, such that the sample or object, or a substructure thereon, can be examined, re-shaped, or otherwise manipulated, in the microchannel of the sample chip.

However, conventional sample chips suffer from the disadvantage that the flow of solution through the microchannels is often too fast in order to isolate or manipulate the particular objects which need to be examined.

Accordingly, a sample chip that includes a working area wherein objects or substructures of objects in a high-speed flow of solution can be isolated, re-shaped, investigated, or manipulated, is needed.

### SUMMARY OF THE INVENTION

The present invention allows a user to precisely hold and move samples, such as microscopic dielectric objects including cells and beads in solution, using focused laser light.

The present invention allows the user to introduce an object into a region of high flow while maintaining the ability to hold, observe, and later collect the object. Thus, in the present invention, a sample chip is used to introduce, hold, and flow solutions containing samples or other materials used in experiments or processing, within a microchannel or within a sample chamber of intersecting microchannels. Laser-generated optical traps are used to extract samples of interest within the microchannel or sample chamber of intersecting microchannels, and allow manipulation of the samples.

In one embodiment of the present invention, the sample chip includes a body portion;

and a cover portion disposed on the body portion; wherein an upper surface of the body portion includes a plurality of microchannels in which objects are introduced for examination and manipulation by optical traps.

In another embodiment of the present invention, at least one of the microchannels or sample chambers includes a barrier which, independently or in combination with optical traps, aligns, supports, holds, or manipulates the obejcts.

The number of microchannels and their configuration can vary, and the microchannels may intersect, the sample chamber being disposed at the intersection of the microchannels.

The barrier includes at least one of a plurality of barrier structures which are integrally formed or removably disposed in the sample chamber. The barrier structures can take different shapes and can be in any combination of shapes.

In one embodiment, a sample chip includes a body portion; and a cover portion disposed on the body portion, such that the body portion and the cover portion form a plurality of microchannels therein; and a sample chamber disposed in at least one of the microchannels, such that the sample chamber in which objects are introduced, is positioned within a working focal region of an apparatus for producing optical traps to for experimentation and manipulation of said objects by said optical traps.

In another embodiment, a sample chip, includes a body portion; and a cover portion disposed on the body portion such that the body portion and the cover portion form a plurality of microchannels therein; and a barrier formed in at least one of the microchannels at a working focal region of an apparatus for producing optical traps.

There has thus been outlined, rather broadly, some features consistent with the present invention in order that the detailed description thereof that follows may be better understood, and in order that the present contribution to the art may be better appreciated. There are, of course, additional features consistent with the present invention that will be described below and which will form the subject matter of the claims appended hereto.

In this respect, before explaining at least one embodiment consistent with the present invention in detail, it is to be understood that the invention is not limited in its application to the details of construction and to the arrangements of the components set forth in the following description or illustrated in the drawings. Methods and apparatuses consistent with the present invention are capable of other embodiments and of being practiced and carried out in various ways. Also, it is to be understood that the phraseology and terminology employed herein, as well as the abstract included below, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the methods and apparatuses consistent with the present invention.

### BRIEF DESCRIPTION OF THE DRAWINGS

FIG. 1 illustrates a cross-sectional side view of a sample chip according to one embodiment consistent with the present invention.

FIG. 2A illustrates a plan view of a sample chip with microchannels according to one embodiment consistent with the present invention.

FIG. 2B (I) and (II) illustrate two plan views of a sample chip with microchannels according to yet other embodiments consistent with the present invention.

FIG. 2C illustrates a plan view of a sample chip with microchannels according to yet another embodiment consistent with the present invention.

FIG. 2D illustrates a plan view of a sample chip with microchannels according to yet another embodiment consistent with the present invention.

FIG. 2E illustrates a plan view of a sample chip with microchannels according to yet another embodiment consistent with the present invention.

FIG. 3 illustrates a plan view of a sample chamber according to one embodiment consistent with the present invention.

FIG. 4 illustrates a plan view of yet another embodiment of the sample chamber consistent with the present invention.

FIG. 5 illustrates a plan view of yet another embodiment of the sample chamber consistent with the present invention.

FIG. 6 illustrates a perspective view of yet another embodiment of the sample chamber consistent with the present invention.

FIG. 7A illustrates a perspective view of yet another embodiment of the sample chamber consistent with the present invention.

FIG. 8 illustrates a perspective view of yet another embodiment of the sample chamber consistent with the present invention.

FIG. 9 illustrates a plan view of a sample chip with microchannels according to yet another embodiment consistent with the present invention.

FIG. 10 illustrates a plan view of a sample chip with microchannels according to yet another embodiment consistent with the present invention.

### DETAILED DESCRIPTION OF THE PREFERRED EMBODIMENTS

The present invention provides a sample chip which is used as part of a system in research, or in a manufacturing or processing environment, for controlling and manipulating small objects using laser-generated optical traps.

As stated above, the generation of optical traps, and arrays of optical traps, for controlling and manipulating small objects, such as biological material, is known in the art. The optical traps may be plural in number and independently movable. In the present invention, by using optical traps, sample objects can be trapped, controlled and manipulated in a microchannel of a sample chip or cell, through which fluid is introduced, such that after manipulation, the sample objects can be released into the flow of fluid and directed into a recovery vessel as desired.

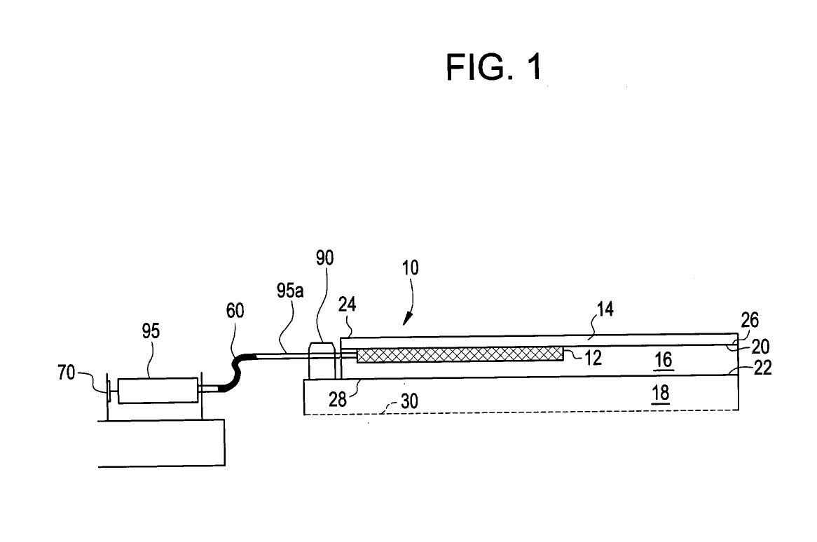

Turning to FIG. 1, a cross-section view of a sample chip or cell 10 is shown. The sample chip or cell 10 typically has a planar or “chip” structure containing two or more separate layers, which when joined together form a plurality of microchannels 12. As shown in FIG. 1, one embodiment of the sample chip 10, includes a cover portion 14, a body portion 16, and in some embodiments, a base portion 18, where the body portion 16 substantially defines the microchannels 12. The body portion 16 includes two surfaces: an upper surface 20 and a lower surface 22. The upper surface 20 of the body portion 16 is fabricated to include grooves and recesses. The cover portion 14 also includes two surfaces: an upper surface 24, and a lower surface 26. The lower surface 26 of the cover portion 14 is joined to the upper surface 20 of the body portion 16, such that the grooves define the microchannels 12 within the sample chip 10. Similarly, the base portion 18 includes and upper surface 28 and a lower surface 30. The upper surface 28 of the base portion 18 is joined to the lower surface 22 of the body portion 16 so that the base portion 18 provides support for the sample chip 10.

The body portion 16, the cover portion 14, and the base portion 18, may be formed of substantially the same or different materials. The material(s) chosen must allow the light which generates the optical traps, to pass into the sample chip 10 and must not otherwise interfere with the formation of the optical traps.

In some embodiments, only the cover portion 14 is transparent to allow laser light for the optical trap to go through the cover portion 14, and the base portion 18 and body portion 16 may be opaque. However, the body portion 16 and the base portion 18 may be preferably transparent to allow for normal bright-field imaging. In other embodiments, the body portion 16 and the base portion 18, if present, are transparent to the laser light while the cover portion 14 may be opaque.

Further, in some embodiments, the location in the sample chip 10 of the objects that are to be controlled and manipulated, is determined by fluorescent methods, and the user might image the objects using fluorescent imaging, which illuminates and images from the direction of the objective lens. In these embodiments, the material(s) used to form either the body portion 16, and the base portion 18, or the cover portion 14, should be transparent to the specific wavelengths used for the fluorescent identification.

The body portion 16 and the cover portion 14 should also be constructed of or coated with a material that is inert to both the objects and the media containing the objects. For example, biological substrates such as cells, proteins, and DNA, should not stick to the surface of the subject sample chip 10, and must not be changed or destroyed by the material.

Similarly, the material should not be degraded under the full range of conditions to which the subject chip 10 might be exposed, including extremes of pH, temperature, and salt concentration.

Additionally, the body portion 16 should be constructed of a material that is compatible with known microfabrication techniques, e.g., photolithography, wet chemical etching, laser ablation, reactive ion etching (RIE), air abrasion techniques, injection molding, LIGA methods, metal electroforming, embossing, and other techniques.

Preferred materials for the body portion 16 include polymeric materials, such as polymethylmethacrylate (PMMA), polycarbonate, or a polysiloxame, such as polydimethylsiloxane (PDMS). Most preferred materials include elastomeric materials such as PDMS.

Pre-formed glass microscope slide coverslips having a thickness of 170 microns are suitable as cover portions 14. Glass microscope slides are suitable as base portions 18. Such coverslips and microscope slides are available from Coming Inc., Greenville, Ohio.

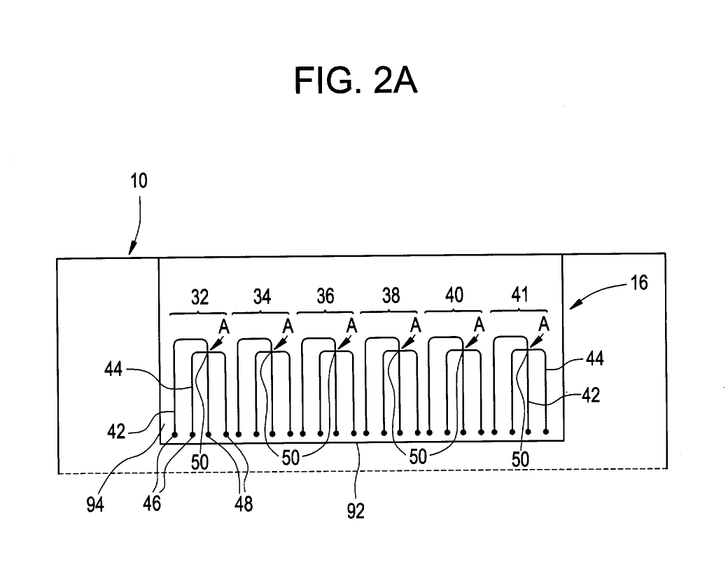

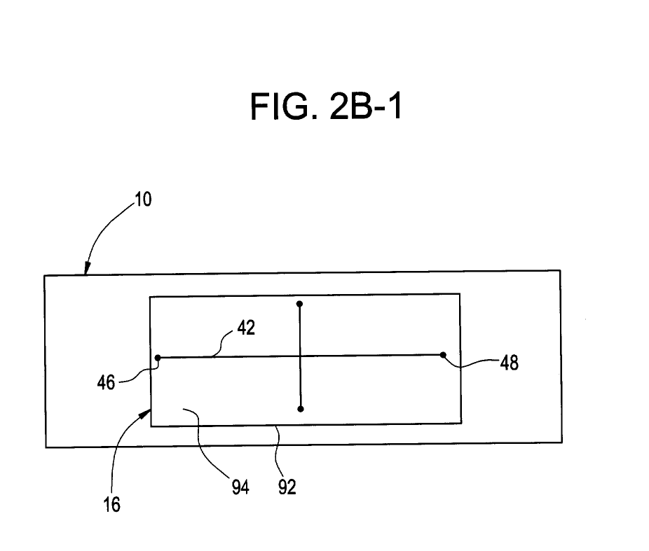

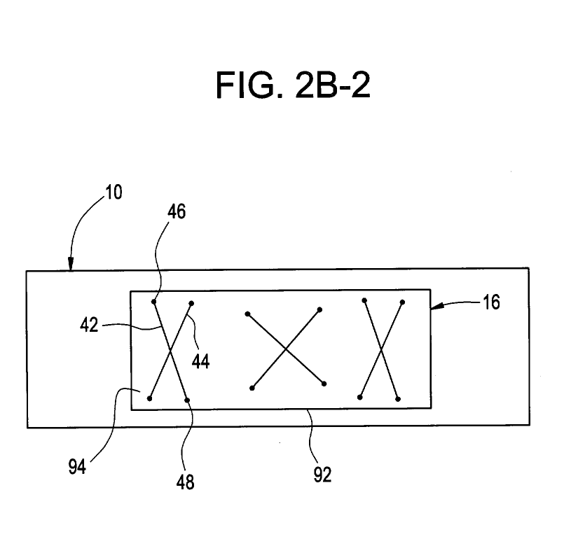

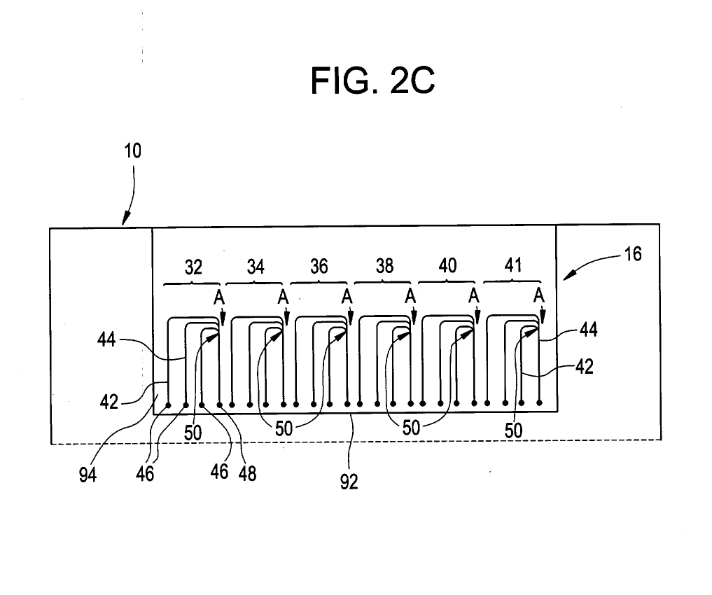

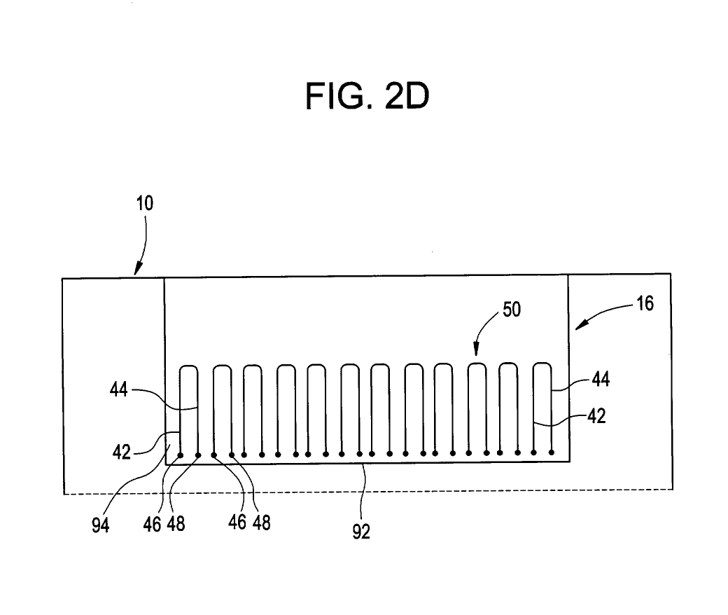

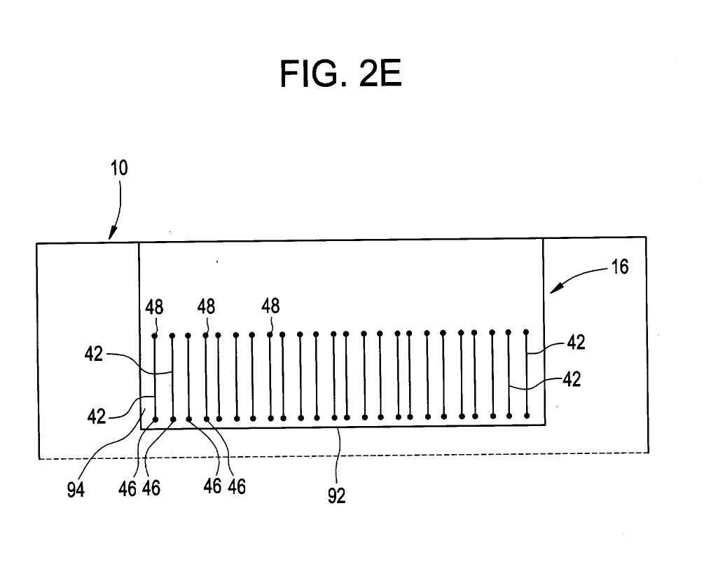

Turning to FIG. 2A, one embodiment of the sample chip 10 is shown in plan view, illustrating a plurality of microchannels 42, 44 that create multiple, independent particle control and manipulation sections 32, 34 36, 38, 40, and 41. Each object control and manipulation section 32, 24, 36, 38, 40, 41 is formed by a pair of U-shaped microchannels 42, 44 (a object supply microchannel 42 and a fluid supply microchannel 44), where each microchannel 42, 44 has an inlet section 46 and an outlet section 48, which are essentially wells.

In those preferred embodiments where the body portion 16 is made of an elastomeric polymer such as PDMS, the inlet sections 46 and outlet sections 48 are aligned and spaced back from one edge 92 of the planar body portion 16, so that the elastomeric material forms a sealing region 94 to protect the inlet sections 46 and outlet sections 48 from contamination and damage until the sample chip 10 is ready for use.

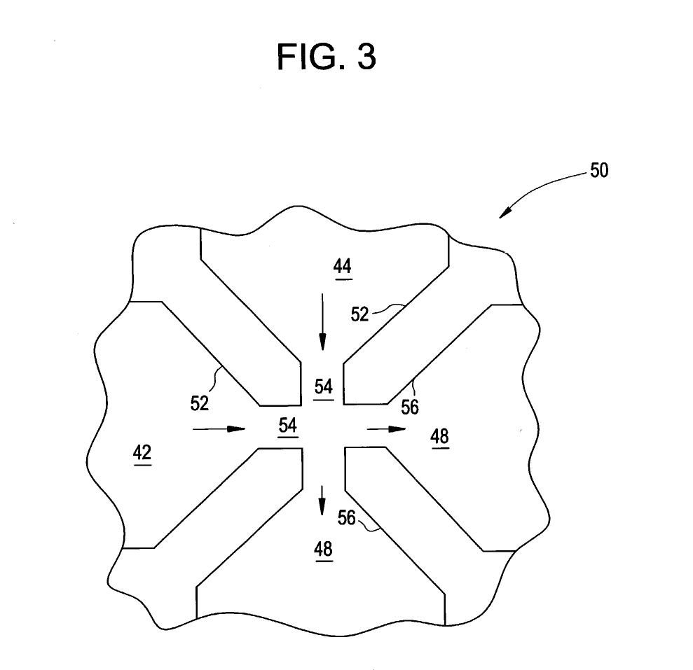

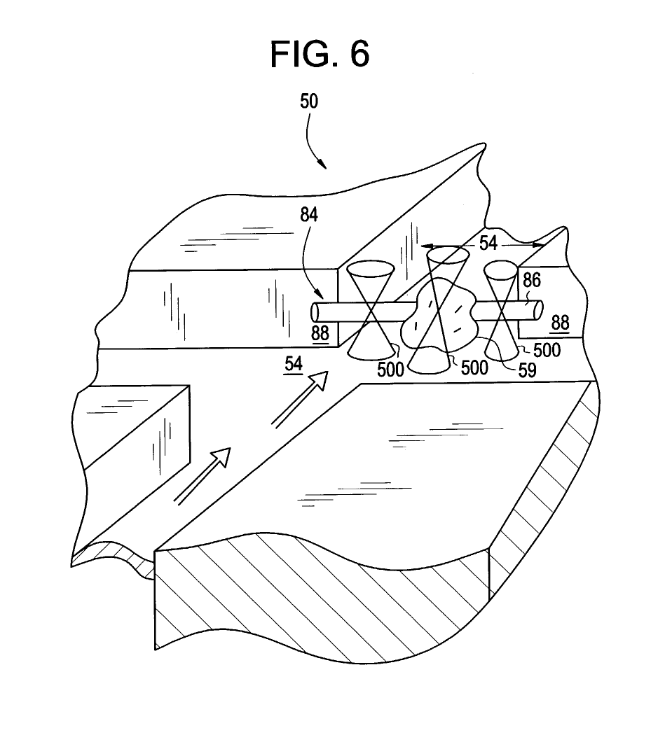

In one embodiment of the invention, each pair of the object supply microchannels 42 and the fluid supply microchannels 44, intersects at a position “A” (see FIG. 2A) to form a unique “M” shape. In one embodiment, the microchannels 42, 44 intersect at 90 degree angles, but a 90 degree angle is not necessary in order for the microchannels to effectively intersect. The intersection A of the microchannels 42, 44 form a region called a sample chamber 50 (see FIG. 3) that can be positioned within the working focal region of an apparatus for producing optical traps (see FIGS. 6-8). The advantage of the sample chamber 50 is that it can be used to manipulate objects using the optical traps, without this manipulation being performed in the microchannel 42, 44 itself. It also allows for two distinct, and independent input flows and two distinct outward flows.

However, the configuration of the microchannels 42, 44 are not necessarily in an “M” shape, but could be configured such that they form a “T” shape or other crossed shapes (see FIG. 2B (I) and (II)). Further, the number of microchannels could be more than four, or the numbers of inlet sections 46 and outlet section 48 could vary in number (i.e., three inlet sections 46 and one outlet section 48, or two inlet section 46 and three outlet section 48 etc.) (see FIG. 2C). Still further, the microchannels could be such that they do not intersect at all, but are disposed next to one another (see FIGS. 2D-E, and discussed below). In such an embodiment, the microchannels can be disposed in any configuration, such as a U-shape (see FIG. 2D), or in parallel lines in the body portion 16 whether vertically, horizontally, or diagonally (see FIG. 2E for a representative drawing).

Further, the sample chamber 50 can be disposed in the microchannels at any point where the working area of the optical traps is located.

An enlarged view of a representative sample chamber 50 is shown in FIG. 3. Typically the inlet sections 46 and the outlet sections 48 of the microchannels 42, 44 have a width of from about 150 microns to about 350 microns, and preferably are about 300 microns. However, the size of the microchannels 42, 44 can vary from several microns or smaller to several millimeters or more.

In one embodiment, the object supply microchannel 42 and the fluid supply microchannel 44 (see FIG. 3) each end in a tapered section 52 that leads to intersecting chamber entrance channels 54. The chamber entrance channels 54 typically have a width of about 50 microns. The chamber entrance channels 54 end in flared sections 56 that lead to the outlet sections 48.

The tapered section 52 exists in order to move from a region of wide channels (i.e., microchannels 42, 44 prior to intersection “A”), where the width of the microchannels 42, 44 minimizes interaction of the objects with the walls of the microchannels 42, 44, and prevents clogging of the microchannels 42, 44, to a region with narrow channels and a small sample chamber 50 (note: the optical size of the sample chamber 50 is typically set by the working area of the apparatus, such as the microscope and the optical trap setup).

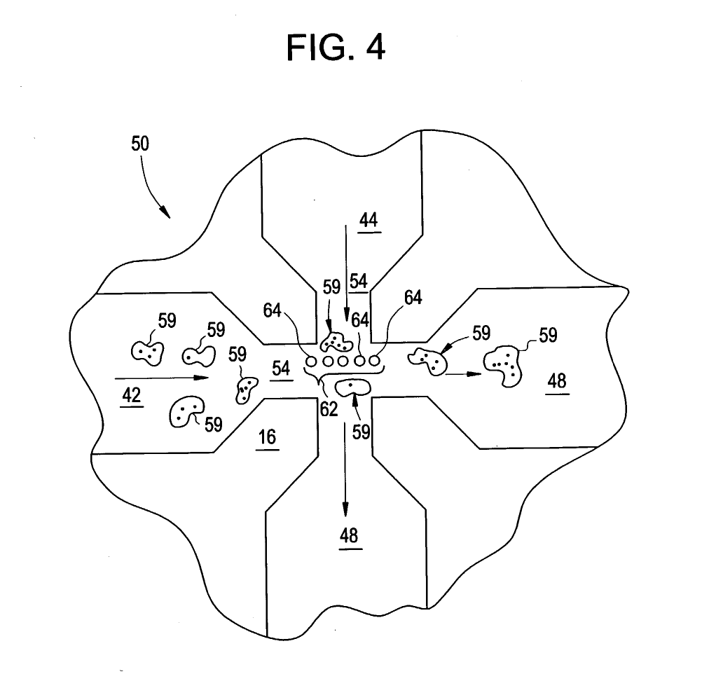

With the tapered section 52, the flow of media or solution through the sample chamber 50 into the chamber entrance channel 54, may occur at a high speed, due to the constriction. However, when the objects enter the sample chamber 50, barriers (see FIG. 4) are placed therein to prevent flow from the top microchannel to the bottom microchannel from dragging the objects 59 to the bottom. Thus, the objects are made available to be held and manipulated by the optical traps.

FIG. 4 is a plan view of one embodiment of a sample chamber 50 having a barrier 62.

The barrier 62 is formed of a series of spaced apart rods 64 that may be integrally formed with the body portion 16, the cover portion (not shown) or both. The spacing of the rods 64 is such that fluid can flow through the barrier 62, but that the objects 59 to be controlled and manipulated cannot. The rods 64 are aligned with the path of the flow of objects 59 through the chamber entrance channel 54 of the object supply microchannel 42 and the rods 64 extend the width of the chamber entrance channel 54 of the fluid supply microchannel 44.

In one embodiment, continuing the idea of the barrier, one or more posts, beads, or other obstacles in a fluidic (sample) chip, would allow fluid or small particles to pass in order to maintain a larger object in an externally applied force, such as that form a fluid flow, electric field, or other externally applied force. Further, a combination of flows and posts in a microfluidic chip can be used to align objects (i.e., a cell with a tail in a solution can flow against the rods 64, and the tails will go through the barrier 62, but the heads do not, leaving the tails to straighten in the flow).

With respect to FIG. 4, in operation, fluid solution is introduced into the sample chamber 50 via, for example, a syringe 95 (see FIG. 1). Alternately, the fluid may be introduced by other means, such as through pipets, open wells, pneumatic pumps, etc. In the embodiment shown in FIG. 4, the arrows indicated the flow of the sample objects 59 and the fluid streams.

With respect to the introduction of the fluid, turning to FIG. 1, in one embodiment, a syringe 95 containing a fluid, is connected to the sample chip 10. As shown in FIG. 1, the base portion 18 extends beyond the sealing region 94 (see FIG. 2A). A needle 95a at one end of the microbore tube 60 penetrates through the sealing region 94 and into one of the inlet sections 46 or outlet sections 48 of the microchannels 42, 44 of the sample chip 10 to be used. An adhesive material 90 is then applied to the syringe needle 95a extending from the body portion 16 to secure the syringe needle 95a to the base portion 18 and body portion 16. The syringe needle 95a connected to the syringe 95 is attached to a microbore tubing 60 which provides a fluid connection between an inlet section 46 and an outlet section 48 and a syringe 95. The syringe 95 is controlled by high precision syringe pumps 70. This is done for both of the inlet sections 46 and outlet sections 48.

To avoid plugging of the syringe needle with the material constructing the sample chip 10 when pushing it through the chip 10, a “non-coring” needle (i.e., one that does not get plugged), such as a “Huber” needle, is used. The Huber needle has a bent tip so that the opening is on the side instead of in the front tip of the needle.

In some embodiments, syringe push-pull pumps 70, which pull fluid from one syringe at an identical rate to that at which it pushes fluid from a second syringe, are employed. In these embodiments, the push-pull pumps 70 are operatively connected to both an inlet section 46 and outlet section 48.

In other embodiments, a common technique called “electro-osmotic flow” or EOF, among other techniques, is used to pump fluid through the microfluidic chip 10. The EOF is performed by applying an electric voltage across the microchannels. In the present invention, the inlet sections 46 would be turned into open wells. The wells are filled with the fluids and the microchannels 42, 44 are primed by pushing fluid through the microchannels 42, 44. Then electrodes (preferably a non-corrosive metal such as platinum) are inserted into each of the four wells 46, 48. The flow rates and directions are controlled by controlling the four lead voltages.

Turning to FIG. 4, note that after the sample objects 59 have been introduced into the sample chamber 50, some of the objects 59 are upstream of the barrier 62 and some are downstream. When the fluid stream is introduced via the syringe 95, the sample objects 59 downstream of the barrier 62 are immediately discharged from the sample chamber 50. The spacing of the rods 64 creating the barrier 62 is chosen so that the sample objects 59 upstream of the barrier 62 cannot pass through. Consequently, the upstream sample objects 59 are held against the barrier 62 and contacted with the fluid.

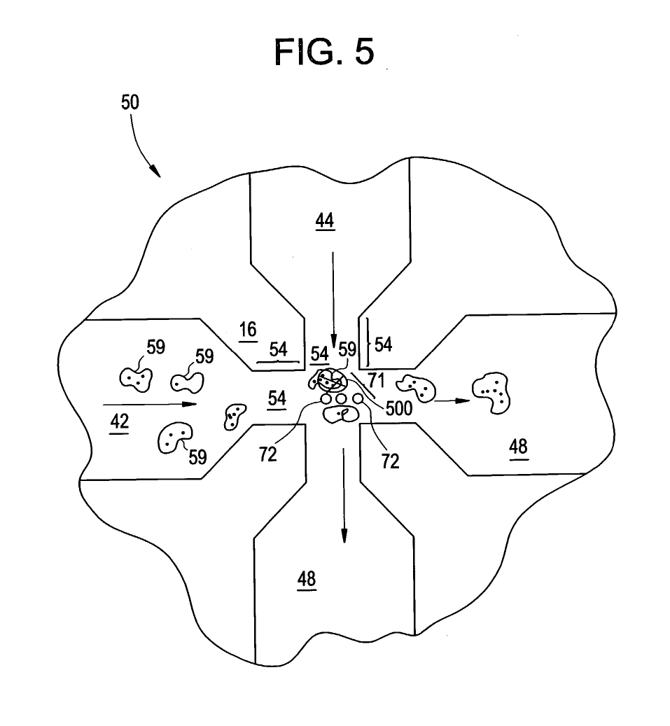

In those embodiments where the base portion 18 is a microscope slide, the sample chip 10 is placed on a microscope through which the optical trap 500 (see FIGS. 5-6) or traps are directed into the sample chamber 50 for use in manipulating sample objects 59 or barrier objects. In a representative method for using the inventive sample chip 10, the object supply inlet channel 42 is primed by introducing a fluid containing sample objects 59 at a relatively fast flow rate, e.g., a flow rate of about 100 microns per second. After priming, the flow rate is adjusted so that the sample objects 59 in the fluid, flow through the object supply entrance channel at a rate of about 10 microns per second and contacts the sample objects 59 at a controlled rate. At this rate, the objects 59 can be held at the barrier 62, trapped, controlled, and manipulated with optical traps using conventional techniques.

Once the sample objects 59 have been contained in the sample chamber 50, the flow rate through the sample object inlet 46 is stopped. Thus, the objects 59 can be moved into the sample chamber 50 by priming the syringe 95, and the flow of solution stopped, or the solution can continue to flow through the sample chamber 50 while the manipulation of the objects 59 takes place. The fluid supply flow rate may be started and increased to a large rate without driving the sample object 59 from the sample chamber 50, as the barrier 62 supports the object 59. Thus, the object 59 may be contacted with the first fluid flows.

After a sufficient period of time of examination and first fluid contact is concluded, the sample objects 59 are released from the optical traps 500 and caused to flow through the fluid supply outlet section 48 into a recovery vessel (not shown). In fact, the objects 59 may be directed to either one outlet section 48 or another depending on whether the recovery vessels hold different types of objects 59.

Thus, the present invention allows an object 59 to be introduced into a region of high flow while maintaining the ability to hold, observe, and later collect the object 59. For example, the user might use an optical trap 500 to hold an object 59 in place while flowing chemicals around it. Then, the user might then flush the first fluid from the microchannel 44 and flow a separate chemical solution around the object 59 to investigate the changes (i.e., a fluorescent label), repeating the process as many times as necessary, or may extract the fluid-contacted object 59 using an optical trap 500, for further study outside of the system (i.e., the fluid supply chamber entrance channel). Note that the present invention preferably has only one of the two microchannels 42, 44 flowing at a time. While optical traps 500 may be used to move objects 59 around when the flow is slow or stationary, only the use of a barrier 62 is strong enough to hold the objects 59 in place when the fast flow occurs.

Thus, the optical trap can hold an object 59 in a flow of a solution around the object 59 to investigate the effect of the solution on the object 59 or to have the solution affect the object 59 in a desired manner.

FIG. 5 illustrates another embodiment of the sample chamber 50 having a barrier 71 in the chamber entrance channel 54 of the sample chamber 50. The barrier 71 is formed of a series of spaced apart rods 72 which may be formed integrally with the body portion 16, the cover portion (not shown), or both, and aligned with the flow path through the chamber entrance channel 54 of the object supply microchannel 42. In the embodiment shown in FIG. 5, the barrier 71 extends along only a portion of the width of the chamber entrance channel 54 of the fluid supply micrchannel 44, instead of along the whole width of the chamber entrance channel 54 as shown in FIG. 4.

In the embodiment shown in FIG. 5, after the barrier 71 is formed, sample objects 59 are introduced into the sample chamber 50. As in FIG. 4, the sample objects 59 downstream of the barrier 71 are immediately discharged from the sample chamber 50. The spacing of the rods 72 creating the barrier 71 is chosen so that the sample objects 59 upstream of the barrier 71 cannot pass through easily. Optical traps 500 are used to position and hold the sample objects 59 against the upstream side of the barrier 71. As stated above with respect to FIG. 4, the fluid is then introduced into the sample chamber 50 and contacted against the thus secured sample objects 59 for a desired time, before the fluid discharges the objects 59 through the outlet 48.

FIG. 6 illustrates a perspective view of another embodiment of a sample chamber 50 having a barrier 84 and operating similarly to that of the apparatus shown in FIG. 5. The barrier 84 is formed of at least one elongated barrier structure 86 of sufficient length so that it can extend across the width 54a of the downstream wall 88 of the chamber entrance channel 54 of the fluid supply microchannel 44 where the microchannels 42, 44 intersect to form the sample chamber 50. In some embodiments, the elongated barrier structure 86 is held in place by one or more optical traps 500 as discussed above with respect to FIG. 5.

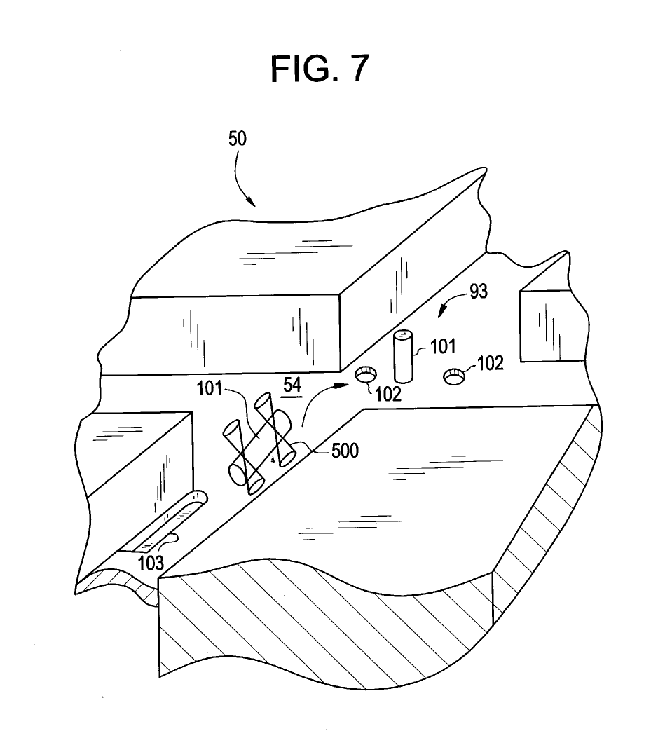

FIG. 7 illustrates a perspective view of another embodiment of a sample chamber 50 operating similarly to the apparatuses shown in FIGS. 5-6, having a barrier 93 formed of a series of spaced apart elongated barrier structures 101 which are removably fitted into barrier recesses 102 in the chamber entrance channel 54 of the fluid supply microchannel 44, and which are oriented perpendicular to the flow of fluid through the microchannel 44. The barrier recesses 102 have perimeters that correspond to the cross-section of at least one end of each of the barrier structures 101. The sample chamber 50 also contains storage recesses 103 (one shown) generally configured to the shape of the elongated barrier objects 101, in which the elongated barrier structures 101 can be stored when the barrier 93 is not needed.

Therefore, the insertion of the barrier structures 101 can be performed in any number found to be convenient, and in any desired configuration. The chamber entrance channel 54 can be pre-formed with recesses 102 in order that the barrier structures 101 can be inserted therein to hold the structures 101 for access by the optical traps 500. The barrier structures 101 can be friction-fitted or force-fitted into the recesses 102, although not so forcefully that they are unable to be removed. An optical vortex can be used to screw the barrier structures 101 in place.

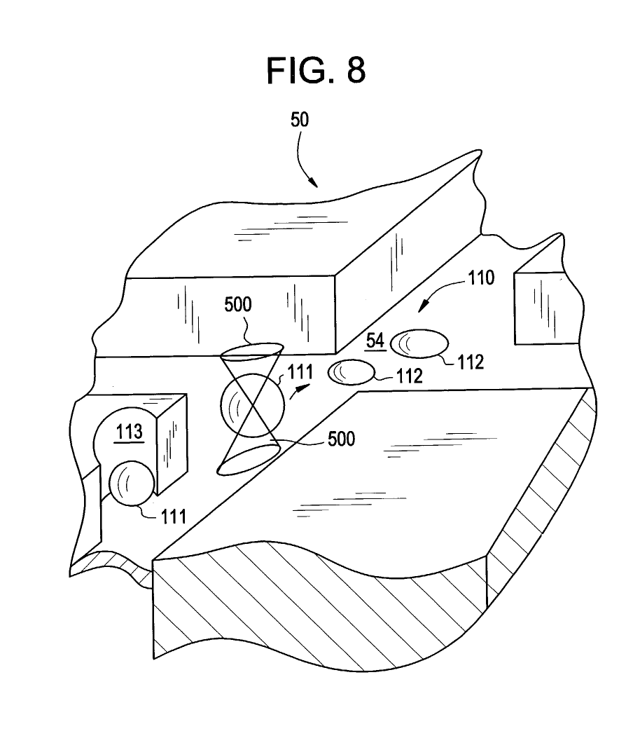

FIG. 8 illustrates a perspective view of another embodiment of a sample chamber 50 operating similarly to that of FIGS. 5-8, having a barrier 110 formed of a series of spaced apart barrier structures 111 which are removably fitted into barrier recesses 112 in the chamber entrance channel 54 of the fluid supply microchannel 44, and which are oriented perpendicular to the flow of fluid through the microchannel 44. The sample chamber 50 also contains storage recesses 113 (one shown) generally configured to the shape of the spherical barrier structures 111, in which the spherical barrier structures 111 can be stored when the barrier 110 is not needed.

The barrier structures 111 are aligned with the path of the flow of objects 59 through the chamber entrance channel 54 of the object supply microchannel 42, and extend for at least a portion of the width of the chamber entrance channel 54 of the fluid supply microchannel 44. The spacing of the barrier structures 111 is such that fluid can flow through the barrier 110, but that the structures 111 to be controlled and manipulated cannot.

In the operation of the apparatus shown in FIG. 8, an optical trap or series of optical traps 500 can trap the spherical barrier structures 111, and transport and then insert the structures 111 into the storage barrier recesses 112. In some embodiments, the optical trap(s) 500 continue to hold the objects 111 once located in the barrier recesses 112 in order to provide additional support to the barrier 110.

The barrier structures 111 are advantageously made of a material that is readily held by the optical trap 500. Suitable materials include, but are not limited to, control pore glass, ceramics, polystyrene, methylstyrene, acrylic polymers, paramagnetic materials, thoriosol, carbon graphite, titanium dioxide, latex, cross-linked dextrans, such as sepharose, cellulose, nylon, cross-linked micelles, Teflon, plastic, diamond, quartz, and silicon.

With respect to the various embodiments of the invention as shown in FIGS. 4-8, the configuration of the barrier structures can be varied depending on the type and number of barrier structures desired. For example, a spherical barrier structure can be used in combination with an elongated barrier structure, etc., such that the barrier is of a desired combination. Accordingly, the barrier structures may all be movable instead of integrally formed with the body portion.

Further, holes or recesses similar to those shown in FIG. 7 can be pre-formed in the body portion, such that beads can be squirted into the chamber entrance channel 54, and moved into the recesses using the optical traps.

Further, the barrier structures may be introduced as objects in a solution. In other embodiments, the barriers may be functionalized to perform specific tasks, such as sticking to certain objects, fluorescing in the presence of certain objects, acting upon objects in certain physical, chemical, biological, in other ways, etc.

In other embodiments of the present invention, as stated above, the microchannels need not intersect, but can be disposed next to one another (see FIGS. 2D-E). In such an embodiment, the microchannels can be disposed in any configuration, such as a U-shape (see FIG. 2D), or in parallel lines in the body portion 16 whether vertically, horizontally, or diagonally (see FIG. 2E for a representative drawing).

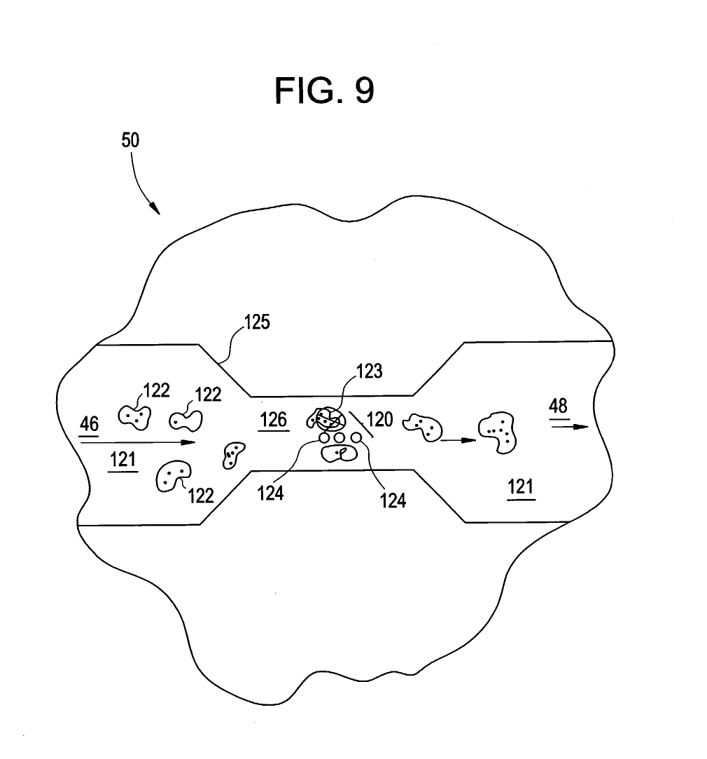

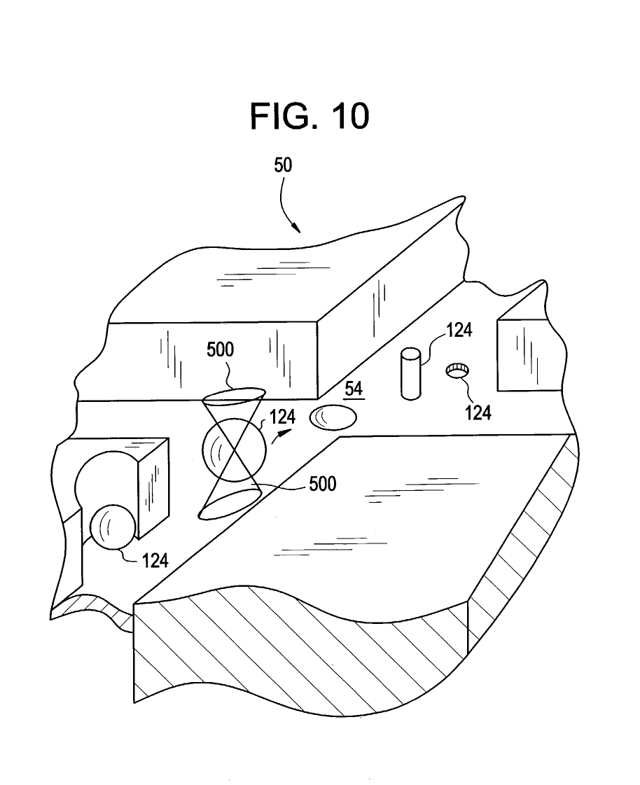

In the embodiment of FIG. 2E, as shown in FIG. 9, the objects 122 are introduced into the inlet sections 46, and barriers 120 are disposed in the microchannels 121 to hold the objects 122 so that the optical traps 123 can manipulate the objects 122 in the microchannel 121. In particular, as discussed above with respect to FIGS. 4-8, the configuration of the barrier structures 124 of the barriers 120 can be varied depending on the type and number of barrier structures 124 desired (see FIG. 10). For example, a spherical barrier structure can be used in combination with an elongated barrier structure, etc., such that the barrier is of a desired combination. Accordingly, the barrier structures 124 may all be movable instead of integrally formed with the body portion 16.

In addition, in one embodiment shown in FIG. 9, the microchannel 121 may also have a tapered section 125 which leads to a sample chamber portion 126 of the microchannel 121, where the optical traps 123 manipulate the objects 122 at the barrier structures 124. After examination of the objects 122, the optical traps 123 release the objects 122 to be discharged through the outlet section 48.

In another embodiment, a sample chamber 50 may be provided with a patterned substrate. The patterning may be in the form of depressions, recesses, holes, wells, slots, ridges, barriers, grooves, pegs, posts or other raised or depressed features. Such patterning may be created using standard photolithographic and other techniques well known in the semiconductor industry including without limitation, etching, depositing, spraying, and sputtering, as well as other techniques commonly used in microfabrication, such as molding, cutting with lasers or tools, melting, abrading, compressing, scraping, drilling, threading, and impacting (such as, without limitation, hammering and stamping).

As shown in FIGS. 7 and 8, in one embodiment, the patterning of the substrate may be employed to help position objects which may be in any shape convenient for interaction with the patterning. For example, without limitation, posts or spheres to interact by insertion into holes, but also flanges to interact by insertion into slots, rounded structures to interact by being cupped by depressions, grooves to orient flat structures parallel with the width of the groove, and variously shaped structures to interact by being channeled by ridges.

In one embodiment, movement of objects through the sample chamber and placement of them in position to interact with the patterning of the substrate may be initiated or maintained with one or any combination of a flow of a fluid (for example, without limitation, a liquid or gas), an electrical, magnetic gravitational, or optical force, or association with a carrier which is moved by such a fluid or force. Positioning of objects within the patterning may be by any one or a combination of a flow of a fluid (for example, without limitation, a liquid or gas), an electrical, magnetic, gravitational or optical force, or association with a tool which is moved by such a fluid or force. Overall, it is preferred that an optical trap be employed for movement or placement.

In one embodiment, objects may be temporarily placed in the patterning or permanently affixed thereto. Examples, without limitation, of placement approaches include friction, crimping, chemical reaction, melting the object or shrinking the feature around the object, magnetic force, electrical force, optical force, suction, and fluid pressure.

In one embodiment, objects, including without limitation, pegs, spheres, and posts, may be provided with a channel, groove or threading to facilitate escape of gas or liquid which might otherwise create back pressure by being trapped beneath the object in the hole.

Since certain changes may be made in the above sample chip without departure from the scope of the invention herein involved, it is intended that all matter contained in the above description, as shown in the accompanying drawings, the specification, and the claims shall be interpreted in an illustrative, and not limiting sense.

What is claimed is:

1. A sample chip, comprising:
a body portion; and
a cover portion disposed on said body portion, such that said body portion and said cover portion form a plurality of microchannels therein; and
a sample chamber into which objects are introduced, said sample chamber being disposed in at least one of said microchannels and positioned within a working focal region of an apparatus for producing optical traps, such that experimentation and manipulation of said objects is performed by said optical traps.

2. The sample chip according to claim 1, wherein said microchannels include at least one pair of object supply microchannels and fluid supply microchannels which intersect at an angle to form said sample chamber.

3. The sample chip according to claim 2, wherein said at least one pair of object supply microchannels and fluid supply microchannels intersect at a 90 degree

4. The sample chip according to claim 1, further comprising:
a base portion on which said body portion is disposed.

5. The sample chip according to claim 4, wherein said cover portion is formed of a transparent material.

6. The sample chip according to claim 5, wherein said body portion and said base portion are formed of a transparent material.

7. The sample chip according to claim 5, wherein said body portion and said base portion are formed of an opaque material.

8. The sample chip according to claim 4, wherein a material of which each said body portion, said cover portion, and said base portion are formed, is the same material.

9. The sample chip according to claim 4, wherein a material of which each said body portion, said cover portion, and said base portion are formed, is different.

10. The sample chip according to claim 9, wherein said transparent material is transparent to a specific wavelength used for fluorescent identification of objects which are introduced into said sample chamber.

11. The sample chip according to claim 1, wherein said body portion and said cover portion are one of constructed of and coated with a material that is inert to objects which are introduced into said sample chamber, and media containing said objects.

12. The sample chip according to claim 1, wherein said body portion is constructed of a material that is compatible with microfabrication techniques, including from a group comprising photolithography, wet chemical etching, laser ablation, reactive ion etching (RIE), air abrasion techniques, injection molding, LIGA methods, metal electroforming, and embossing.

13. The sample chip according to claim 1, wherein said body portion is constructed of a polymeric material, including from a group comprising a polymethylmethacrylate (PMMA), a polycarbonate, and a polysiloxane.

14. The sample chip according to claim 13, wherein said polysiloxane is polydimethylsiloxane (PDMS).

15. The sample chip according to claim 1, wherein said cover portion is a preformed glass microscope slide coverslip.

16. The sample chip according to claim 15, wherein said glass microsphere slide coverslip has a thickness of 170 microns.

17. The sample chip according to claim 4, wherein said base portion is a glass microscope slide.

18. The sample chip according to claim 1, wherein each of said microchannels has an inlet section and an outlet section.

19. The sample chip according to claim 2, wherein each said pair of said object supply microchannels and said fluid supply microchannels has an inlet section and an outlet section.

20. The sample chip according to claim 19, wherein each said pair of object supply microchannels and fluid supply microchannels form independent object control and manipulation sections.

21. The sample chip according to claim 20, wherein each said pair of object supply microchannels and fluid supply microchannels intersect to form a substantial “M” shape.

22. The sample chip according to claim 18, wherein a number of said inlet sections and a number of said outlet sections differ.

23. The sample chip according to claim 18, wherein said microchannels are disposed in said body portion in a substantial “T” shape.

24. The sample chip according to claim 18, wherein said microchannels are disposed parallel to one another.

25. The sample chip according to claim 18, wherein said microchannels are disposed in a curved shape proximate to one another.

26. The sample chip according to claim 18, wherein each said inlet section and said outlet section are aligned and spaced back from one edge of said body portion, forming a sealing region to protect said inlet section and said outlet section from contamination and damage.

27. The sample chip according to claim 18, wherein each said inlet section and said outlet section have a width in a range of 150 to 350 microns.

28. The sample chip according to claim 19, wherein each said object supply channel and said fluid supply channel include a tapered section which leads to a chamber entrance channel of said sample chamber.

29. The sample chip according to claim 28, wherein said chamber entrance channel has a width of about 50 microns.

30. The sample chip according to claim 28, wherein said chamber entrance channel ends in flared sections which lead to said outlet section of said object supply microchannel and said outlet section of said fluid supply microchannel.

31. The sample chip according to claim 18, further comprising:
a chamber entrance channel disposed within said sample chamber of each said microchannels.

32. The sample chip according to claim 19, further comprising:
a chamber entrance channel disposed within said sample chamber, at an intersection of said object supply microchannel and said fluid supply microchannel.

33. The sample chip according to claim 31 or claim 32, further comprising:
a barrier formed in said chamber entrance channel of said sample chamber.

34. The sample chip according to claim 33, wherein said barrier is formed of at least one of a plurality of barrier structures.

35. The sample chip according to claim 34, wherein said barrier structures are spaced apart rods formed integrally with at least one of said body portion and said cover portion.

36. The sample chip according to claim 34, wherein said barrier structures are spaced apart rods removably fitted into said chamber entrance channel.

37. The sample chip according to claim 33, wherein fluid introduced into said sample chamber can flow through said barrier, but objects introduced into said sample chamber cannot flow through said barrier.

38. The sample chip according to claim 34, wherein said barrier structures are aligned with a path of objects through said chamber entrance channel of each of said microchannels.

39. The sample chip according to claim 35, wherein said rods are aligned with a path of objects through said chamber entrance channel of each of said object supply microchannels.

40. The sample chip according to claim 35, wherein said rods extend along at least a portion of a width of said chamber entrance channel of said fluid supply microchannel.

41. The sample chip according to claim 34, wherein said rods extend along at least a portion of a width of said chamber entrance channel of each of said microchannels.

42. The sample chip according to claim 4, wherein fluid is introduced into said sample chamber via one of a syringe and by EOF.

43. The sample chip according to claim 42, wherein said syringe is driven by a motor.

44. The sample chip according to claim 43, wherein an adhesive material is applied to a needle of said syringe which extends from said body portion, to secure said needle to said base portion and said body portion.

45. The sample chip according to claim 42, wherein a flow rate of said fluid is about 100 microns per second.

46. The sample chip according to claim 19, wherein said optical traps hold said objects and direct said objects to at least one of a predetermined outlet section of said microchannels.

47. The sample chip according to claim 32, wherein said optical traps can move said objects outside of said chamber entrance channel, such that a first fluid can be flushed from said fluid supply microchannel and replaced with a second fluid, and said objects can be placed into contact with said second fluid using said optical traps.

48. The sample chip according to claim 33, wherein said optical traps position and hold objects introduced into said sample chamber, against an upstream side of said barrier.

49. The sample chip according to claim 33, wherein said barrier is formed of at least one elongated barrier structure of a length which extends horizontally across a width of opposing downstream walls of said chamber entrance channel.

50. The sample chip according to claim 49, wherein said elongated barrier structure is held in place by at least one optical trap.

51. The sample chip according to claim 34, further comprising at a plurality of barrier recesses in which said barrier structures are fitted to be oriented perpendicular to a flow through said microchannels.

52. The sample chip according to claim 51, further comprising:
at least one storage recess which is generally configured to a shape of at least one of said barrier structures, said at least one storage recess in which said one of said barrier structures can be stored when said barrier is not needed.

53. The sample chip according to claim 52, wherein said barrier structures are elongated in shape.

54. The sample chip according to claim 52, wherein said barrier structures are spherical in shape.

55. The sample chip according to claim 34, wherein said barrier structures are held in place by at least one optical trap.

56. The sample chip according to claim 55, wherein said barrier structures are made of a material from a group comprising control pore glass, ceramics, polystyrene, methylstyrene, acrylic polymers, paramagnetic materials, thoriosol, carbon graphite, titanium oxide, latex, cross-linked dextrans, nylon, cross-linked micelles, plastic, diamond, quartz, and silicon.

57. The sample chip according to claim 52, wherein said barrier structures are a combination of different shapes.

58. The sample chip according to claim 57, wherein said barrier structures are removably fitted into said barrier recesses.

59. The sample chip according to claim 1, wherein said microchannels are formed by a plurality of grooves disposed in an upper surface of said body portion.

60. The sample chip according to claim 6, wherein said cover portion is formed of an opaque material.

61. The sample chip according to claim 34, wherein said barrier structures allow a predetermined size of said objects to pass said barrier, and hold objects of a size greater than said predetermined size at said barrier by an externally applied force.

62. The sample chip according to claim 61, wherein said externally applied force is an electric field.

63. The sample chip according to claim 34, wherein said barrier structures are used to align said objects in a flow in said microchannels.

64. The sample chip according to claim 44, wherein said needle is a non-coring needle.

65. The sample chip according to claim 45, wherein said fluid one of has an effect on said objects in said microchannels and has a predetermined effect on said objects.

66. The sample chip according to claim 51, wherein chamber entrance channel is preformed with one of said barrier recesses and beads.

67. The sample chip according to claim 51, wherein an optical vortex is used to insert said barrier structures into said barrier recesses.

68. The sample chip according to claim 34, wherein said barrier structures are introduced as objects into said sample chamber.

69. The sample chip according to claim 33, wherein said barrier is functionalized to perform specific tasks, including adhering to predetermined of said objects introduced into said sample chamber, fluorescing in a presence of said objects, and acting upon said objects in one of physical, chemical, and biological ways.

70. The sample chip according to claim 1, wherein said microchannels include a tapered section which leads to said sample chamber.

71. The sample chip according to claim 1, wherein said sample chamber is provided with a patterned substrate.

72. The sample chip according to claim 71, wherein said patterned substrate is at least one of a group including depressions, recesses, holes, wells, slots, ridges, barriers, grooves, pegs, posts, raised and depressed features.

73. The sample chip according to claim 72, wherein said patterning is created using photolithographic techniques.

74. The sample chip according to claim 72, wherein said patterned substrate assists in the positioning of said objects in said sample chamber.

75. The sample chip according to claim 72, wherein said objects are positioned to interact with said patterned substrate by at least one of an electrical, magnetic, gravitational, or optical force.

76. The sample chip according to claim 75, wherein said positioning is one of permanent and temporary.

77. The sample chip according to claim 51, wherein said objects are provided with a groove to facilitate escape of one of gas and liquid when said objects are positioned in said recesses.

78. A sample chip, comprising:
a cover portion disposed on said body portion, such that said body portion and said cover portion form a plurality of microchannels therein, into which objects are introduced; and
a barrier formed in at least one of said microchannels at a working focal region of an apparatus for producing optical traps such that said objects are examined and manipulated using said optical traps.

79. The sample chip according to claim 78, further comprising:
a barrier formed in said one of said microchannels.

80. The sample chip according to claim 79, wherein said barrier is formed of at least one of a plurality of barrier structures.

81. The sample chip according to claim 80, wherein said barrier structures are spaced apart rods formed integrally with at least one of said body portion and said cover portion.

82. The sample chip according to claim 80, wherein said barrier structures are spaced apart rods removably fitted in said one of said microchannels.

83. The sample chip according to claim 33, wherein fluid introduced into said microchannels can flow through said barrier, but said objects introduced into said microchannels cannot flow through said barrier.

84. The sample chip according to claim 83, wherein said barrier structures are aligned with a path of said objects flowing through said microchannels.

85. The sample chip according to claim 84, wherein barrier structures extend along at least a portion of a width of each of said microchannels.

86. The sample chip according to claim 80, wherein said barrier structures include at least one elongated barrier structure of a length which extends horizontally across a width of opposing downstream walls of each of said microchannels.

87. The sample chip according to claim 86, wherein said elongated barrier structure is held in place by at least one of said optical traps.

88. The sample chip according to claim 80, further comprising at a plurality of barrier recesses in which said barrier structures are fitted to be oriented perpendicular to a flow through said microchannels.

89. The sample chip according to claim 88, further comprising:
at least one storage recess which is generally configured to a shape of at least one of said barrier structures, said at least one storage recess in which said one of said barrier structures can be stored when said barrier is not needed.

90. The sample chip according to claim 89, wherein said barrier structures are elongated in shape.

91. The sample chip according to claim 89, wherein said barrier structures are spherical in shape.

92. The sample chip according to claim 80, wherein said barrier structures are held in place by at least one of said optical traps.

93. The sample chip according to claim 80, wherein said barrier structures are made of a material from a group comprising control pore glass, ceramics, polystyrene, methylstyrene, acrylic polymers, paramagnetic materials, thoriosol, carbon graphite, titanium oxide, latex, cross-linked dextrans, nylon, cross-linked micelles, plastic, diamond, quartz, and silicon.

94. The sample chip according to claim 89, wherein said barrier structures are a combination of different shapes.

95. The sample chip according to claim 88, wherein said barrier structures are removably fitted into said barrier recesses.

96. The sample chip according to claim 80, wherein said barrier structures allow a predetermined size of objects introduced into said microchannels to pass said barrier, and hold objects of a size greater than said predetermined size at said barrier by an externally applied force.

97. The sample chip according to claim 96, wherein said externally applied force is an electric field.

98. The sample chip according to claim 83, wherein said fluid one of has an effect on said objects introduced into said microchannels and has a predetermined effect on said objects.

99. The sample chip according to claim 88, wherein an optical vortex is used to insert said barrier structures into said barrier recesses.

100. The sample chip according to claim 79, wherein said barrier structures are introduced as objects into said microchannels.

101. The sample chip according to claim 79, wherein said barrier is functionalized to perform specific tasks, including adhering to predetermined of said objects introduced into said microchannels, fluorescing in a presence of said objects, and acting upon said objects in one of physical, chemical, and biological ways.

102. The sample chip according to claim 88, wherein said objects introduced into said microchannels are provided with a groove to facilitate escape of one of gas and liquid when said objects are positioned in said recesses.
